(19) United States
(12) Patent Application Publication
(10) Pub. No.: US 2005/0214860 A1
(43) Pub. Date: Sep. 29, 2005

(54) Antibodies specific to KDR and uses thereof

(76) Inventors: Zhenping Zhu, Saddle Brook, NJ (US); Larry Witte, Stormville, NY (US)

Correspondence Address: KENYON & KENYON, ONE BROADWAY, NEW YORK, NY, 10004, US

(21) Appl. No.: 10/809,387

(22) Filed: Mar. 23, 2004

Related U.S. Application Data
(63) Continuation of application No. 09/976,787, filed on Oct. 12, 2001, now abandoned.
Application No. 09/976,787 is a continuation of application No. 09/493,539, filed on Jan. 28, 2000, now abandoned.
(60) Provisional application No. 60/117,726, filed on Jan. 29, 1999.

(51) Int. Cl. G01N 33/53; C07K 16/40

(52) U.S. Cl. 435/7.1; 530/388.26

(57) ABSTRACT

The invention provides an immunoglobulin molecule which binds KDR with an affinity comparable to human VEGF, and that neutralizes activation of KDR. Immunoglobulin molecules include monovalent single chain antibodies, multivalent single chain antibodies, diabodies, triabodies, antibodies, humanized antibodies and chimerized antibodies. The invention further provides nucleic acid molecules that encode these immunoglobulin molecules. The invention also provides a method of making the immunoglobulin molecules mentioned above. The invention further provides a method of neutralizing the activation of KDR, a method of inhibiting angiogenesis in a mammal and a method of inhibiting tumor growth in a mammal with such immunoglobulin molecules.

### BACKGROUND OF THE INVENTION

Angiogenesis is the process of developing new blood vessels. The process involves the proliferation, migration and tissue infiltration of capillary endothelial cells from pre-existing blood vessels. Angiogenesis is important in normal physiological processes including embryonic development, follicular growth, and wound healing as well as in pathological conditions involving tumor growth and metastasis. (Folkman, J. and Klagsbrun, M. Science 235:442-447 (1987)).

The vascular endothelium is usually quiescent in adults, and its activation is tightly regulated during angiogenesis. Several factors have been implicated as possible regulators of angiogenesis in vivo. These include transforming growth factor (TGFb), acidic and basic fibroblast growth factor (aFGF and bFGF), platelet derived growth factor (PDGF), and vascular endothelial growth factor (VEGF) (Klagsbrun, M. and D'Amore, P. (1991) Annual Rev. Physiol. 53: 217-239). VEGF, an endothelial cell-specific mitogen, is distinct among these factors in that it acts as an angiogenesis inducer by specifically promoting the proliferation of endothelial cells.

VEGF is a homodimeric glycoprotein consisting of two 23 kD subunits with structural similarity to PDGF. Four different monomeric isoforms of VEGF exist resulting from alternative splicing of mRNA. The isoforms include two membrane bound forms (VEGF₂₀₆ and VEGF₁₈₉) and two soluble forms (VEGF₁₆₅ and VEGF₁₂₁). In all human tissues except placenta, VEGF₁₆₅ is the most abundant isoform.

VEGF is expressed in embryonic tissues (Breier et al., Development (Camb.) 114:521 (1992)), macrophages, proliferating epidermal keratinocytes during wound healing (Brown et al., J. Exp. Med., 176:1375 (1992)), and may be responsible for tissue edema associated with inflammation (Ferrara et al., Endocr. Rev. 13:18 (1992)). In situ hybridization studies have demonstrated high VEGF expression in a number of human tumor lines including glioblastoma multiforme, hemangioblastoma, central nervous system neoplasms and AIDS-associated Kaposi's sarcoma (Plate, K. et al. (1992) Nature 359: 845-848; Plate, K. et al. (1993) Cancer Res. 53: 5822-5827; Berkman, R. et al. (1993) J. Clin. Invest. 91: 153-159; Nakamura, S. et al. (1992) AIDS Weekly, 13 (1)). High levels of VEGF were also observed in hypoxia induced angiogenesis (Shweiki, D. et al. (1992) Nature 359: 843-845).

The biological response of VEGF is mediated through high affinity VEGF receptors, which are selectively expressed on endothelial cells during both embryogenesis (Millauer, B., et al. (1993) Cell 72: 835-846) and tumor formation. VEGF receptors typically are class III receptor-type tyrosine kinases characterized by having several, typically five or seven, immunoglobulin-like loops in their amino-terminal extracellular receptor ligand-binding domains (Kaipainen et al., J. Exp. Med. 178:2077-2088 (1993)). The other two regions include a transmembrane region and a carboxy-terminal intracellular catalytic domain interrupted by an insertion of hydrophilic interkinase sequences of variable lengths, called the kinase insert domain (Terman et al., Oncogene 6:1677-1683 (1991). VEGF receptors include FLT-1, sequenced by Shibuya M. et al., Oncogene 5, 519-524 (1990); KDR (kinase insert domain-containing receptor), described in PCT/US92/01300, filed Feb. 20, 1992, and in Terman et al., Oncogene 6:1677-1683 (1991); and FLK-1, sequenced by Matthews W. et al. Proc. Natl. Acad. Sci. USA, 88:9026-9030 (1991). KDR is the human homolog of the mouse FLK-1 receptor. The KDR and FLK-1 receptors are also known as VEGFR2.

Equivalent receptors having homologous amino acid sequences, as defined above, occur in all mammals, e.g. human, mouse. The binding of an antibody to one VEGF receptor does not necessarily imply binding to another VEGF receptor, and binding to a VEGF receptor in one mammal does not necessarily imply binding to the equivalent receptor in another mammal.

It has been suggested that the KDR pathway plays an important role in human tumor angiogenesis. VEGF receptors are expressed mainly on endothelial cells and hematopoietic cells. After VEGF is synthesized and secreted by tumor cells, VEGF binds to VEGFR2 receptors and stimulates the growth of new blood vessels. It has been found that tumors are incapable of growing beyond a certain size unless they have a dedicated blood supply.

The production of monoclonal antibodies against the murine VEGF receptor, FLK-1, by hybridoma technology was described in PCT application US95/01678. These monoclonal antibodies were said to inhibit receptor activation by interferring with the interaction between VEGF and its receptor. It was demonstrated that these antibodies specifically inhibit VEGF stimulation of FLK-1 receptor expressed on transfected 3T3 cells. In vivo it was shown that the antibodies strongly inhibited the growth of human glioblastoma xenografts in nude mice by inhibiting tumor associated angiogenesis.

Not all antibodies that bind to the murine receptor, FLK-1, however, also react with its human homologue, KDR, with sufficient affinity to be commercially viable products for human therapy. Therefore, anti-KDR antibodies having higher affinity than those known in the prior art are needed.

The object of the present invention is to provide highly effective antibodies that neutralize the interaction between VEGF and its human receptor, KDR, with affinities higher than those known in the prior art.

### SUMMARY OF THE INVENTION

These and other objects, as will be apparent to those having ordinary skill in the art, have been met by providing an immunoglobulin molecule which binds KDR with an affinity comparable to human VEGF, and that neutralizes activation of KDR.

Immunoglobulin molecules include monovalent single chain antibodies, multivalent single chain antibodies, diabodies, triabodies, antibodies, humanized antibodies and chimerized antibodies.

The invention further provides nucleic acid molecules that encode these immunoglobulin molecules.

The invention also provides a method of making the immunoglobulin molecules mentioned above. The invention further provides a method of neutralizing the activation of KDR, a method of inhibiting angiogenesis in a mammal and a method of inhibiting tumor growth in a mammal with such immunoglobulin molecules.

Abbreviations

VEGF, vascular endothelial growth factor; bFGF, basic fibroblast growth factor; KDR, kinase insert domain-containing receptor (also known as VEGF receptor 2); FLK-1, fetal liver kinase 1; scFv, single chain Fv; HUVEC, human umbilical vein endothelial cells; PBS, 0.01M phosphate buffered saline (pH 7.2); PBST, PBS containing 0.1% Tween-20; AP, alkaline phosphatase; EGF, epidermal growth factor; V_{H} and V_{L}, variable domain of immunogloblin heavy and light chain, respectively

### DESCRIPTION OF THE FIGURES

FIG. 1 is a graph showing the direct binding of different scFv antibodies (p1C11, p1F12, p2A6 and p2A7) to immobilized KDR.

FIG. 2 is a graph showing the inhibition of binding of KDR to immobilized VEGF₁₆₅ by different scFv antibodies (p1C11, p1F12, p2A6 and p2A7).

FIG. 3 is a graph showing the inhibition of VEGF-induced HUVEC proliferation by scFv antibodies (p2A6 and p1C11).

FIG. 4 is the nucleotide and deduced amino acid sequence of V_{H} and V_{L} chains of c-p1C11.

FIG. 5 is a graph showing the direct binding of antibodies (c-p1C11, p1c11, p2A6) to immobilized KDR.

FIG. 6 is a graph showing the FACS analysis of c-p1C11 binding to KDR-expressing HUVEC.

FIG. 7 is a graph showing the inhibition of binding of KDR receptor to immobilized VEGF₁₆₅ by different scFv antibodies (c-p1C11, p1C11, p2A6).

FIG. 8 is a graph showing the inhibition of binding of radiolabeled VEGF₁₆₅ to immobilized KDR receptor by c-p1C11 and cold VEGF₁₆₅.

FIG. 9 is a graph showing the inhibition of VEGF-induced HUVEC proliferation by anti-KDR antibodies (c-p1C11, p1C11).

### DETAILED DESCRIPTION OF THE INVENTION

The present invention provides immunoglobulin molecules which bind specifically to an extracellular domain of KDR with an affinity comparable to that of VEGF. As a result of such binding, the immunoglobulin molecules are able to neutralize activation of the receptor more effectively than immunoglobulin molecules previously described. An extracellular domain of a VEGF receptor is herein defined as a ligand-binding domain on extracellular region of the receptor.

Immunoglobulin molecules are proteins that recognize and bind to a specific antigen or substance. Immunoglobulin molecules include naturally occuring antibodies, monovalent single chain antibodies, multivalent single chain antibodies, diabodies, triabodies, chimerized antibodies, humanized antibodies and other molecules which bind specifically with antigens.

The immunoglobulin molecules of the present invention bind KDR with an affinity comparable to that of the natural ligand. Affinity, represented by the equilibrium constant for the association of an antigen with an immunoglobulin molecule (K), measures the binding strength between an antigenic determinant and an immunoglobulin molecule, irrespective of the number of binding sites. An antigenic determinant, also known as an epitope, is the site on an antigen at which a given immunoglobulin molecule binds. Typical values of K are 10⁵ to 10¹¹ liters/mol. Any K less than 10⁴ liters/mol is considered to indicate binding which is nonspecific. The reciprocal of K is designated as K_{d}. (K_{d} also may be referred to as the dissociation constant.) The lesser the value of the K_{d}, the stronger the binding strength between an antigenic determinant and the antibody binding site.

The natural ligand of KDR is human VEGF. VEGF binds KDR with the affinity (K_{d}) of 0.93 nM. In order to hinder the binding of VEGF with KDR, an anti-KDR antibody needs to bind KDR with an affinity comparable to VEGF. In other words, the anti-KDR antibody needs to successfully compete with VEGF with respect to binding KDR. An antibody with a K_{d} of at most 5 nM is considered to bind with an affinity comparable to the natural ligand. The antibodies of the invention preferably bind KDR with an affinity of at most about 4 nM, more preferably with an affinity of at most about 3 nM, most preferably with an affinity of at most about 2 nM, and optimally with an affinity of about 1 nM.

The antibodies of the invention neutralize KDR. (See Examples.) In this specification, neutralizing a receptor means diminishing and/or inactivating the intrinsic kinase activity of the receptor to transduce a signal. A reliable assay for KDR neutralization is the inhibition of receptor phosphorylation.

The present invention is not limited by any particular mechanism of KDR neutralization. At the time of filing this application, the mechanism of KDR neutralization by antibodies is not well understood, and the mechanism followed by one antibody is not necessarilly the same as that followed by another antibody. Some possible mechanisms include preventing binding of the VEGF ligand to the extracellular binding domain of the KDR, and preventing dimerization or oligomerization of receptors. Other mechanisms cannot, however, be ruled out.

A preferred immunoglobulin molecule is an antibody fragment called a monovalent single chain antibody (scFv). Monovalent single chain antibodies include an antibody variable heavy-chain fragment (V_{H}) linked to an antibody variable light-chain fragment (V_{L}) by a peptide linker which allows the two fragments to associate to form a functional antigen binding site (see, for example U.S. Pat. No. 4,946,778, Ladner et al., (Genex); WO 88/09344, Creative Biomolecules, Inc/Huston et al.). WO 92/01047, Cambridge Antibody Technology et al./McCafferty et al., describes the display of scFv fragments on the surface of soluble recombinant genetic display packages, such as bacteriophage. A single chain antibody with a linker (L) can be represented as V_{L}-L-V_{H} or V_{H}-L-V_{L}.

Valency refers to the number of antigen binding sites which an immunoglobulin molecule has for a particular epitope. For example, a monovalent antibody has one binding site for a particular epitope. Avidity is the measure of the strength of binding between an immunoglobulin molecule with its antigen. Avidity is related to both the affinity between an epitope with its antigen binding site on the immunoglobulin molecule, and the valency of the immunoglobulin molecule.

Single chain antibodies lack some or all of the constant domains of the whole antibodies from which they are derived. Therefore, they may overcome some of the problems associated with the use of whole antibodies. For example, single-chain antibodies tend to be free of undesired interactions between biological molecules and the heavy-chain constant region, or other unwanted biological activity. Additionally, single-chain antibodies are considerably smaller than whole antibodies and may therefore have greater capillary permeability than whole antibodies, allowing single-chain antibodies to localize and bind to target antigen-binding sites more efficiently. Also, single chain antibodies can be produced on a relatively large scale in prokaryotic cells, thus facilitating their production. Furthermore, the relatively small size of single-chain antibodies makes them less likely to provoke an unwanted immune response in a recipient than whole antibodies.

The peptide linkers used to produce the single chain antibodies may be flexible peptides selected to assure that the proper three-dimensional folding of the V_{L} and V_{H} domains may occur once they are linked so as to maintain the target molecule binding-specificity of the full length anti-KDR antibody. Generally, the carboxyl terminus of the V_{L} or V_{H} sequence may be covalently linked by such a peptide linker to the amino acid terminus of a complementary V_{H} or V_{L} sequence. The linker is generally 10 to 50 amino acid residues. Preferably, the linker is 10 to 30 amino acid residues. More preferably the linker is 12 to 30 amino acid residues. Most preferably is a linker of 15 to 25 amino acid residues. An example of such linker peptides include (Gly-Gly-Gly-Gly-Ser)₃.

Single chain antibodies, each having one V_{H} and one V_{L} domain covalently linked by a first peptide linker, can be covalently linked by at least one more peptide linker to form a multivalent single chain antibody. Multivalent single chain antibodies allow for the construction of antibody fragments which have the specificity and avidity of whole antibodies, but lack the constant regions of the full length antibodies.

Multivalent immunoglobulin molecules may be monospecific or multispecific. The term specificity refers to the number of different types of antigenic determinants to which a particular immunoglobulin molecule can bind. If the immunoglobulin molecule binds to only one type of antigenic determinant, the immunoglobulin molecule is monospecific. If the immunoglobulin molecule binds to different types of antigenic determinants then the immunoglobulin molecule is multispecific.

For example, a bispecific multivalent single chain antibody allows for the recognition of two different types of epitopes. The epitopes may both be on KDR. Alternatively, one epitope may be on KDR, and the other epitope may be on another antigen.

Each chain of a multivalent single chain antibody includes a variable light-chain fragment and a variable heavy-chain fragment, and is linked by a peptide linker to at least one other chain. The peptide linker is composed of at least fifteen amino acid residues. The maximum number of amino acid residues is about one hundred. In a preferred embodiment, the number of V_{L} and V_{H} domains is equivalent. Preferably, the peptide linker (L₁) joining the V_{H} and V_{L} domains to form a chain and the peptide linker (L₂) joining two or more chains to form a multivalent scFv have substantially the same amino acid sequence.

For example, a bivalent single chain antibody can be represented as follows:
V_{L}-L₁-V_{H}-L₂-V_{L}-L₁-V_{H} or V_{L}-L₁-V_{H}-L₂-V_{H}-L₁-V_{L} or V_{H}-L₁-V_{L}-L₂-V_{H}-L₁-V_{L} or
V_{H}-L₁-V_{L}-L₂-V_{L}-L₁-V_{H}.

Multivalent single chain antibodies which are trivalent or greater have one or more antibody fragments joined to a bivalent single chain antibody by additional peptide linkers. One example of a trivalent single chain antibody is:
V_{L}-L₁-V_{H}-L₂-V_{L}-L₁-V_{H}-L₂-V_{L}-L₁-V_{H}.

Two single chain antibodies can be combined to form diabodies, also known as bivalent dimers. Diabodies have two chains. Each chain of the diabody includes a V_{H} domain connected to a V_{L} domain. The domains are connected with linkers that are short enough to prevent pairing between domains on the same chain, thus driving the pairing between complementary domains on different chains to recreate the two antigen-binding sites. The peptide linker includes at least five amino acid residues and no more than ten amino acid residues, e.g. (Gly-Gly-Gly-Gly-Ser), (Gly-Gly-Gly-Gly-Ser)₂. The diabody structure is rigid and compact. The antigen-binding sites are at opposite ends of the molecule. Diabodies may be monospecfic or bispecific.

Three single chain antibodies can be combined to form triabodies, also known as trivalent trimers. Triabodies are constructed with the amino acid terminus of a V_{L} or V_{H} domain directly fused to the carboxyl terminus of a V_{L} or V_{H} domain, i.e., without any linker sequence. The triabody has three Fv heads with the polypeptides arranged in a cyclic, head-to-tail fashion. A possible conformation of the triabody molecule is planar with the three binding sites located in a plane at an angle of 120 degrees from one another. Triabodies may be monospecific, bispecific or trispecific.

Preferably the antibodies of this invention contain all six complementarity determining regions of the whole antibody, although antibodies containing fewer than all of such regions, such as three, four or five CDRs, are also functional.

In order to minimize the immunogenicity of murine antibodies, the present invention also provides chimerized and humanized antibodies which bind specifically to an extacellular domain of KDR and neutralize activation of the receptor. Chimeric antibodies include variable regions from the mouse antibody and constant regions from a human antibody. A chimeric antibody retains its binding specificity but more closely resembles a natural human antibody. The only part of humanized antibodies which contain mouse amino acids are those which are necessary to recognize KDR, i.e. the CDRs.

DNA encoding chimerized antibodies may be prepared by recombining DNA substantially or exclusively encoding human constant regions and DNA encoding variable regions derived substantially or exclusively from the sequence of the variable region of a mammal other than a human.

DNA encoding humanized antibodies may be prepared by recombining DNA encoding constant regions and variable regions, other than the CDRs, derived substantially or exclusively from the corresponding human antibody regions and DNA encoding CDRs derived substantially or exclusively from a mammal other than a human.

Suitable mammals, other than a human, include any mammal from which monoclonal antibodies may be made. Examples of such mammals include a rabbit, rat, mouse, horse, goat, or primate. Mice are preferred.

Suitable sources of DNA molecules that encode fragments of antibodies include any cell, such as hybridomas and spleen cells, that express the full length antibody. Another source is single chain antibodies produced from a phage display library as is known in the art.

The antibodies of this invention may be or may combine members of any immunoglobulin class, such as IgG, IgM, IgA, IgD, or IgE, and the subclasses thereof.

The KDR used to make the immunoglobulin molecule is usually bound to a cell, such as an endothelial cell. The KDR may also be bound to a non-endothelial cell, such as a tumor cell. Alternatively, the KDR may be free from the cell, preferably in soluble form.

In the examples below high affinity anti-KDR scFv antibodies, which block VEGF binding to KDR, were isolated from a phage display library constructed from mice immunized with a soluble form of the human VEGF receptor. Over 90% of recovered clones after two rounds of selection are specific to KDR. The binding affinities for KDR of these scFvs are in the nM range, which are as high as those of several bivalent anti-KDR monoclonal antibodies produced using hybridoma technology.

A human phage library may also be used to generate such high affinity anti-KDR scFvs.

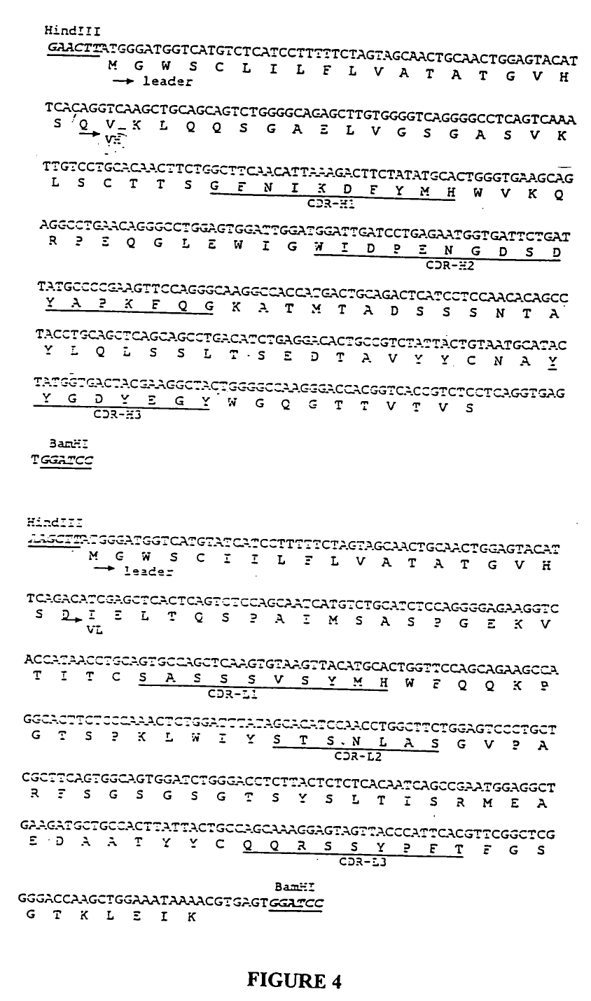

The scFv antibody, p1C11 (FIG. 4), was produced from a single chain (scFv) antibody isolated from a phage display library. (See Examples below.) p1C11 was shown to block VEGF-KDR interaction and inhibit VEGF-stimulated receptor phosphorylation and mitogenesis of HUVEC. This scFv binds both soluble KDR and cell surface-expressed KDR on HUVEC. p1C11 is one of the preferred scFvs of this invention. It binds to KDR with high affinity (K_{d}=2.1 nM).

Each domain of the antibodies of this invention may be a complete immunoglobulin heavy or light chain variable domain, or it may be a functional equivalent or a mutant or derivative of a naturally occuring domain, or a synthetic domain constructed, for example, in vitro using a technique such as one described in WO 93/11236 (Medical Research Council et al./Griffiths et al.). For instance, it is possible to join together domains corresponding to antibody variable domains which are missing at least one amino acid. The important characterizing feature is the ability of each domain to associate with a complementary domain to form an antigen binding site. Accordingly, the terms “variable heavy/light chain fragment” should not be construed to exclude variants which do not have a material effect on how the invention works.

Functional equivalents of the invention include polypeptides with amino acid sequences substantially the same as the amino acid sequence of the variable or hypervariable regions of the full length KDR antibodies. “Substantially the same” amino acid sequence is defined herein as a sequence with at least 70%, preferably at least about 80%, and more preferably at least about 90% homology to another amino acid sequence, as determined by the FASTA search method in accordance with Pearson and Lipman, Proc. Natl. Acad. Sci. USA 85, 2444-2448 (1988).

The antibodies of this invention may be fused to additional amino acid residues. Such residues may be a peptide tag, perhaps to facilitate isolation, or they may be a signal sequence for secretion of the polypeptide from a host cell upon synthesis. Suitably, secretory leader peptides are used, being amino acids joined to the N-terminal end of a polypeptide to direct movement of the polypeptide out of the cytosol.

The present invention also provides nucleic acid molecules which comprise a sequence encoding a polypeptide according to the invention, and diverse repertoires of such nucleic acid.

The DNA deletions and recombinations described above may be carried out by known methods, such as those described in PCT applications WO 93/21319, WO 89/09622, European Patent applications 239,400, 338,745 and 332,424 and/or other standard recombinant DNA techniques, such as those described below.

In the examples below chimeric anti-KDR antibody, c-p1C11, was produced from p1c11. Chimeric-p1C11 binds specifically to the extracellular domain of soluble as well as cell surface-expressed KDR. The binding affinity of the c-p1C11 is 0.82 nM. It effectively neutralizes activation of KDR and MAP kinases p44/p42 of human endothelial cells. Furthermore, c-p1C11 efficiently neutralizes VEGF-induced mitogenesis of human endothelial cells.

The c-p1C11 binds more efficiently to KDR than its parent scFv, p1C11, and is more potent in neutralizing activity of KDR interaction and in inhibiting VEGF-stimulated mitogenesis of HUVEC. The affinity of the c-p1C11 for binding to KDR is approximately 2.5-fold higher than that of the parent scFv, mainly due to the slower off-rate of the bivalent c-p1C11 (See Table 2.)

### EXAMPLES

The Examples which follow are set forth to aid in understanding the invention but are not intended to, and should not be construed to, limit its scope in any way. The Examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989) Molecular Cloning: A Laboratory Manual, 2nd edition, Cold Spring Harbor Laboratory Press.

### Example I

### Producing Single Chain Antibodies

### Example I(a)

### Cell Lines and Proteins

Primary-cultured HUVEC was maintained in EBM-2 medium at 37° C., 5% CO₂. Cells were used between passage 2-5 for all assays. VEGF₁₆₅ protein was expressed in baculovirus and purified. cDNA encoding the extracellular domain of KDR was isolated by RT-PCR from human fetal kidney mRNA and subcloned into the Bgl II and BspE I sites of the vector AP-Tag. In this plasmid the cDNA for KDR extracellular domain is fused in-frame with the cDNA for human placental AP. The plasmid was electroporated into NIH 3T3 cells together with the neomycin expression vector pSV-Neo and stable cell clones were selected with G418. The soluble fusion protein KDR-AP was purified from cell culture supernatant by affinity chromatography using immobilized monoclonal antibodies to AP.

### Example I(b)

### Mice Immunization and Construction of Single Chain Antibody Phage Display Library

Female BALB/C mice were given two intraperitoneal (i.p.) injections of 10 ug KDR-AP in 200 ul of RIBI Adjuvant System followed by one i.p. injection without RIBI adjuvant over a period of two months. The mice were also given a subcutaneous (s.c.) injection of 10 ug KDR-AP in 200 ul of RIBI at the time of the first immunization. The mice were boosted i.p. with 20 ug of KDR-AP three days before euthanasia. Spleens from donor mice were removed and the cells were isolated. RNA was extracted and mRNA was purified from total RNA of splenocytes. A scFv phage display library was constructed using the mRNA which was displayed on the surface of the filamentous phage M13.

In displaying the scFv on filamentous phage surface, antibody V_{H} and V_{L} domains are joined together by a 15 amino-acid-long linker (GGGGS)³ and fused to the N-terminal of phage protein III. A 15 amino-acid-long E tag, which is followed by an amber codon (TAG), was inserted between the C-terminal of V_{L} and the protein III for detection and other analytic purposes. The amber codon positioned between the E tag and the protein III enables the construct to make scFv in surface-displaying format when transformed into a suppressor host (such as TGI cells), and scFv in soluble form when transformed into a nonsupressor host (such as HB2151 cells).

The assembled scFv DNA was ligated into the pCANTAB 5E vector. The transformed TG1 cells were plated onto 2YTAG plates and incubated. The colonies were scraped into 10 ml of 2YT medium, mixed with 5 ml 50% glycerol and stored at −70 C as the library stock.

### Example I(c)

### Biopanning

The library stock was grown to log phase, rescued with M13K07 helper phage and amplified overnight in 2YTAK medium (2YT containing 100 ug/ml of ampicillin and 50 ug/ml of kanamycin) at 30 C. The phage preparation was precipitated in 4% PEG/0.5M NaCl, resuspended in 3% fat-free milk/PBS containing 500 ug/ml of AP protein and incubated at 37 C for 1 h to capture phage displaying anti-AP scFv and to block other nonspecific binding.

KDR-AP (10 ug/ml) coated Maxisorp Star tubes (Nunc, Denmark) were first blocked with 3% milk/PBS at 37 C for 1 h, and then incubated with the phage preparation at room temperature for 1 h. The tubes were washed 10 times with PBST followed by 10 times with PBS (PBS containing 0.1% Tween 20). The bound phage was eluted at room temperature for 10 min. with 1 ml of a freshly prepared solution of 100 mM triethylamine. The eluted phage were incubated with 10 ml of mid-log phase TG1 cells at 37 C for 30 min. stationary and 30 min. shaking. The infected TG1 cells were then plated onto 2YTAG plates and incubated overnight at 30 C.

Ninety-nine percent (185/186) of clones screened after the 3rd round of panning were found to be specific KDR binders. However, only 15 (8%) of these binders could block KDR binding to immobilized VEGF. DNA BstN I fingerprinting of these 15 clones indicated the presence of 2 different digestion patterns; whereas 21 randomly picked VEGF nonblockers yielded 4 different patterns. All the digestion patterns were also seen in clones identified after the 2nd round of panning. Representative clones of each digestion pattern were picked from clones recovered after the 2nd round of panning and subject to DNA sequencing. Out of 15 clones sequenced, 2 unique VEGF blockers and 3 nonblockers were identified. One scFv, p2A7, which neither binds to KDR nor blocks VEGF binding to KDR, was selected as a negative control for all studies.

### Example I(d)

### Phage ELISA

Individual TG1 clones were grown at 37 C in 96 well plates and rescued with M13K07 helper phage as described above. The amplified phage preparation was blocked with ⅙ volume of 18% milk/PBS at RT for 1 h and added to Maxi-sorp 96-well microtiter plates (Nunc) coated with KDR-AP or AP (1 ug/ml×100 ul). After incubation at room temperature for 1 h, the plates were washed 3 times with PBST and incubated with a rabbit anti-M13 phage Ab-HRP conjugate. The plates were washed 5 times, TMB peroxidase substrate added, and the OD at 450 nm read using a microplate reader and scFv antibodies were identified and sequenced.

### Example I(e)

### Preparation of Soluble scFv

Phage of individual clones were used to infect a nonsuppressor E. coli host HB2151 and the infectant selected on 2YTAG-N plates. Expression of scFv in HB2 151 cells was induced by culturing the cells in 2YTA medium containing 1 mM isopropyl-1-thio-B-D-galactopyranoside at 30 C. A periplasmic extract of the cells was prepared by resuspending the cell pellet in 25 mM Tris (pH 7.5) containing 20% (w/v) sucrose, 200 mM NaCl, 1 mM EDTA and 0.1 mM PMSF, followed by incubation at 4 C with gentle shaking for 1 h. After centrifugation at 15,000 rpm for 15 min., the soluble scFv was purified from the supernatant by affinity chromatography using the RPAS Purification Module (Pharmacia Biotech).

### Example II

### Assays

### Example II(a)

### Quantitative KDR Binding Assay

Two assays were employed to examine quantitatively the binding of purified soluble scFv to KDR.

Four different clones, including the two VEGF blockers, p1C11 and p1F12, one nonblocker, the dominant clone p2A6 and the nonbinder p2A7, were expressed in shaker flasks using a nonsuppressor host E. coli HB2151 cells. The soluble scFv was purified from the periplasmic extracts of E. coli by anti-E-tag affinity chromatography. The yield of purified scFv of these clones ranged from 100-400 ug/liter culture.

In the direct binding assay, various amounts of soluble scFv were added to KDR-coated 96-well Maxi-sorp microtiter plates and incubated at room temerature for 1 h, after which the plates were washed 3 times with PBST. The plates were then incubated at room temerature for 1 h with 100 ul of mouse anti-E tag antibody followed by incubation with 100 μl of rabbit anti-mouse antibody-HRP conjugate. The plates were washed and developed following the procedure described above for the phage ELISA.

In another assay, i.e., the competitive VEGF blocking assay, various amounts of soluble scFv were mixed with a fixed amount of KDR-AP (50 ng) and incubated at room temerature for 1 h. The mixture were then transferred to 96-well microtiter plates coated with VEGF₁₆₅ (200 ng/well) and incubated at room temperature for an additional 2 h, after which the plates were washed 5 times and the substrate for AP was added to quantify the bound KDR-AP molecules. IC₅₀, i.e., the scFv concentration required for 50% inhibition of KDR binding to VEGF, was then calculated.

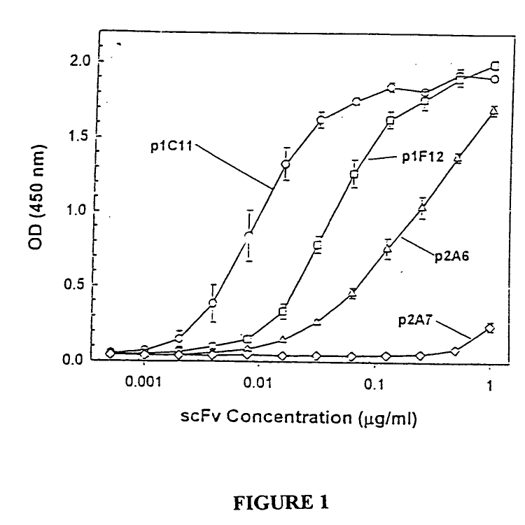

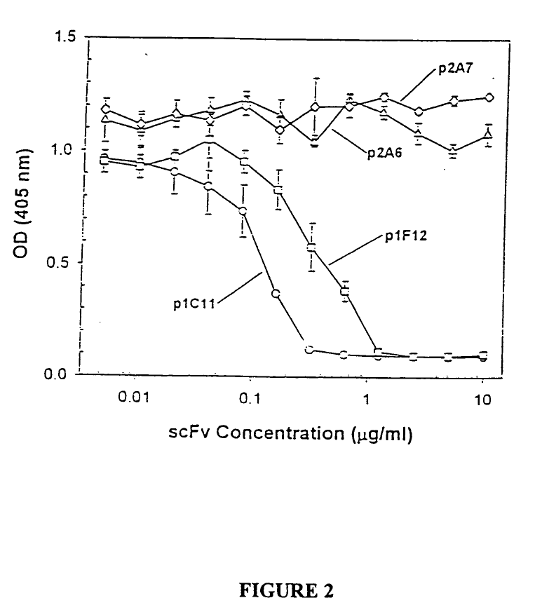

FIG. 1 shows the dose-dependent binding of scFv to immobilized KDR as assayed by a direct binding ELISA. Clone p1C11 and p1F12, but not p2A6, also block KDR binding to immobilized VEGF as shown in FIG. 2. Data shown in FIG. 2 are the means±SD of triplicate determinations. The negative control clone, p2A7, did not bind to KDR nor block KDR binding to VEGF (FIGS. 1 and 2). Clone p1C11, the dominant clone after each round of panning, showed the highest KDR binding capacity and the highest potency in blocking VEGF binding to KDR (Table 1). The antibody concentrations of clone p1C11 required for 50% of maximum binding to KDR (FIG. 1) and for 50% of inhibition of KDR binding to VEGF (FIG. 2) were 0.3 nM and 3 nM, respectively (See Table 1). FACS analysis demonstrated that p1C11, p1F12 and p2A6 were also able to bind to cell surface expressed receptor on HUVEC.

### Example II(b)

### BIAcore Analysis of the Soluble scFv

The binding kinetics of soluble scFv to KDR were measured using BIAcore biosensor (Pharmacia Biosensor). KDR-AP fusion protein was immobilized onto a sensor chip and soluble scFv were injected at concentrations ranging from 62.5 nM to 1000 nM. Sensorgrams were obtained at each concentration and were evaluated using a program, BIA Evaluation 2.0, to determine the rate constant kon and koff. Kd was calculated from the ratio of rate constants koff/kon.

Table 1 shows the results of the surface plasmon resonance on a BIAcore instrument. The VEGF-blocking scFv, p1C11 and p1F12, bound to immobilized KDR with Kd of 2.1 and 5.9 nM, respectively. The non-blocking scFv, p2A6, bound to KDR with approximately a 6-fold weaker affinity (Kd, 11.2 nM) than the best binder p1C11, mainly due to a much faster dissociation rate. As anticipated, p2A7 did not bind to the immobilized KDR on the BIAcore.

### Example II(c)

### Phosphorylation Assay

Phosphorylation assays were performed with early passage HUVEC following a protocol described previously. Briefly, HUVEC were incubated in serum free EBM-2 base medium supplemented with 0.5% bovine serum albumin at room temperature for 10 min. in the presence or absence of scFv antibodies at 5 ug/ml, followed by stimulation with 20 ng/ml VEGF₁₆₅ at room temperature for an additional 15 min. The cells were lysed and the KDR receptor was immunoprecipitated from the cell lysates with Protein A Sepharose beads coupled to a rabbit anti-KDR polyclonal antibody (ImClone Systems Incorporated). The beads were washed, mixed with SDS loading buffer, and the supernatant subjected to Western blot analysis. To detect KDR phosphorylation, blots were probed with an anti-phosphotyrosine Mab, 4G10. For the MAP kinase activity assay, cell lysates were resolved with SDS-PAGE followed by Western blot analysis using a phospho-specific MAP kinase antibody. All signals were detected using ECL.

Results showed that VEGF-blocking scFv p1C11, but not the non-blocking scFv p2A6, was able to inhibit KDR receptor phosphorylation stimulated by VEGF. Further, p1C11 also effectively inhibited VEGF-stimulated activation of MAP kineses p44/p42. In contrast, neither p1C11, nor p2A6 inhibited FGF-stimulated activation of MAP kinases p44/p42.

### Example II(d)

### Anti-Mitogenic Assay

HUVEC (5×103 cells/well) were plated onto 96-well tissue culture plates (Wallach, Inc., Gaithersburg, Md.) in 200 ul of EBM-2 medium without VEGF, bFGF or EGF and incubated at 37 C for 72 h. Various amounts of antibodies were added to duplicate wells and pre-incubated at 37 C for 1 h, after which VEGF₁₆₅ was added to a final concentration of 16 ng/ml. After 18 h of incubation, 0.25 uCi of [3H]-TdR (Amersham) was added to each well and incubated for an additional 4 h. The cells were placed on ice, washed twice with serum-containing medium, followed by a 10 minute incubation at 4 C with 10% TCA. The cells were then washed once with water and solubilized in 25 ul of 2% SDS. Scintillation fluid (150 ul/well) was added and DNA incorporated radioactivity was determined on a scintillation counter (Wallach, Model 1450 Microbeta Scintillation Counter).

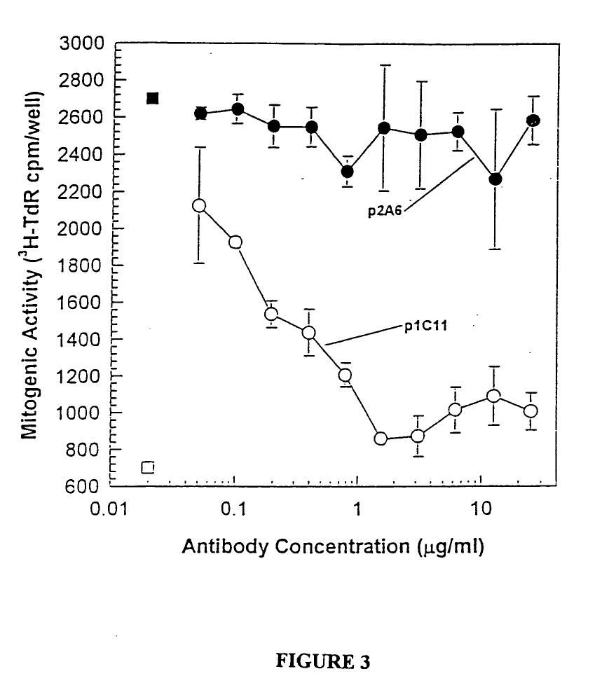

The ability of scFv antibodies to block VEGF-stimulated mitogenic activity on HUVEC is shown in FIG. 3. The VEGF-blocking scFv p1C11 strongly inhibited VEGF induced DNA synthesis in HUVEC with an EC₅₀, i.e., the antibody concentration that inhibited 50% of VEGF-stimulated mitogenesis of HUVEC, of approximately 5 nM. The non-blocking scFv p2A6 showed no inhibitory effect on the mitogenic activity of VEGF. Neither p1C11 nor p2A6 inhibited bFGF-induced DNA synthesis in HUVEC (not shown). Data shown in FIG. 3 are representative of at least three separate experiments. ( ) VEGF only; ( ) no VEGF.

### Example IV

### Producing Chimeric Antibodies

### Example IV(a)

### Cell Lines and Proteins

Primary-cultured human umbilical vein endothelial cells (HUVEC) were maintained in EBM-2 medium at 37 C, 5% CO₂. Cells between passage 2-5 were used for all assays. VEGF₁₆₅ and KDR-alkaline phosphatase fusion proteins (KDR-AP) were expressed in baculovirus and NIH 3T3 cells, respectively, and purified following the procedures described above. The anti-KDR scFv p1C11 and scFv p2A6, an antibody that binds to KDR but does not block KDR-VEGF interaction, were isolated from a phage display library constructed from a mouse immunized with KDR as described above. C225 is a chimeric IgG1 antibody directed against epidermal growth factor (EGF) receptor.

### Example IV(b)

### Cloning of the Variable Domains of scFv p1C11

The variable domains of the light (V_{L}) and the heavy (V_{H}) chains of p1C11 were cloned from the scFv expression vector by PCR using primers 1 and 2, and primers 3 and 4, respectively. The leader peptide sequence for protein secretion in mammalian cells was then added to the 5′ of the V_{L} and the V_{H} by PCR using primers 5 and 2, and primers 5 and 4, respectively.

| | |
|---|---|
| Primer 1: | 5′ CTA GTA GCA ACT GCA ACT GGA GTA CAT TCA GAC ATC |
| | GAG CTC3′ |
| Primer 2: | 5′ TCG ATC TAG AAG GAT CCA CTC ACG TTT TAT TTC CAG3′ |
| | BamHI |
| Primer 3: | 5′ CTA GTA GCA ACT GCA ACT GGA GTA CAT TCA CAG GTC |
| | AAG CTG3′ |
| Primer 4: | 5′ TCG AAG GAT CCA CTC ACC TGA GGA GAC GGT3′ |
| | BamHI |
| Primer 5: | 5′ GGT CAA AAG CTT ATG GGA TGG TCA TGT ATC ATC CTT TTT |
| | Hind III |
| | CTA GTA GCA ACT3′ |

### Example IV(c)

### Construction of the Expression Vectors for the Chimeric p1C11 IgG

Separate vectors for expression of chimeric IgG light chain and heavy chains were constructed. The cloned V_{L} gene was digested with Hind III and BamHI and ligated into the vector pKN100 containing the human κ light chain constant region (C_{L}) to create the expression vector for the chimeric p1C11 light chain, c-p1C11-L. The cloned V_{H} gene was digested with Hind III and BamH I and ligated into the vector pGID105 containing the human IgG1 (γ) heavy chain constant domain (C_{H}) to create the expression vector for the chimeric p1C11 heavy chain, c-p1C11-H. Both constructs were examined by restriction enzyme digestion and verified by dideoxynucleotide sequencing.

As seen in FIG. 4 both the V_{H} and the V_{L} domains are precisely fused on their 5′ ends to a gene segment encoding a leader peptide sequence as marked. The V_{H} and the V_{L} domains are ligated via Hind I/BamH I sites into expression vector pGID105, which contains a cDNA version of the human γ1 constant region gene, and pKN100, which contains a cDNA version of the human κ chain constant region gene, respectively. In each case, expression is under control of the HCMVi promoter and terminated by an artificial termination sequence. The light and the heavy chain complimentarily determining region (CDR) residues, defined according the hypervariable sequence definition of Kabat et al., are underlined and labeled CDR-H1 to H3 and CDR-L1 to L3, respectively.

### Example IV(d)

### IgG Expression and Purification

COS cells were co-transfected with equal amounts of c-p1C11-L and c-p1C11-H plasmids for transient IgG expression. Subconfluent COS cells grown in DMEM/10% FCS in 150 mm culture dishes were rinsed once with 20 ml of DMEM containing 40 mM Tris (pH 7.4), followed by incubation at 37 C for 4.5 h with 4 ml of DMEM/DEAE-Dextran/DNA mixture (DMEM containing 40 mM Tris, 0.4 mg/ml of DEAE-Dextran (Sigma), and 20 ug each of c-p1C11-L and c-p1C11-H plasmids). The cells were incubated at 37 C for 1 h with 4 ml of DMEM/2% FCS containing 100 nM of chloroquine (Sigma), followed by incubation with 1.5 ml of 20% glycerol/PBS at room temperature for 1 min. The cells were washed twice with DMEM/5% FCS and incubated in 20 ml of the same medium at 37 C overnight. The cell culture medium was changed to serum-free DMEM/HEPES after the cells were washed twice with plain DMEM. The cell culture supernatant was collected at 48 h and 120 h after the transfection. The chimeric IgG was purified from the pooled supernatant by affinity chromatography using Protein G column following the protocol described by the manufacturer (Pharmacia Biotech). The IgG-containing fractions were pooled, buffer exchanged into PBS and concentrated using Centricon 10 concentrators (Amicon Corp., Beverly, Mass.). The purity of the IgG was analyzed by SDS-PAGE. The concentration of purified antibody was determined by ELISA using goat anti-human y chain specific antibody as the capture agent and HRP-conjugated goat anti-human k chain antibody as the detection agent. Standard curve was calibrated using a clinical grade antibody, C225.

After affinity purification by Protein G, a single protein band of ˜150 kD was seen in SDS-PAGE. Western blot analysis using HRP-conjugated anti-human IgG1 Fc specific antibody confirmed the presence of human IgG Fc portion in the purified protein (not shown).

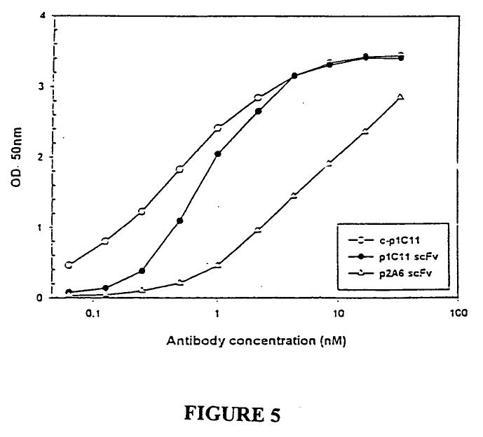

The results of the ELISA show that c-p1C11 binds more efficiently to immobilized KDR than the parent scFv (FIG. 5).

### Example V

### Assays and Analysis

### Example V(a)

### FACS Analysis

Early passage HUVEC cells were grown in growth factor-depleted EBM-2 medium overnight to induce the expression of KDR. The cells were harvested and washed three times with PBS, incubated with c-p1C11 IgG (5 ug/ml) for 1 h at 4 C, followed by incubation with a FITC labeled rabbit anti-human Fc antibody (Capper, Organon Teknika Corp., West Chester, Pa.) for an additional 60 min. The cells were washed and analyzed by a flow cytometer (Model EPICS®, Coulter Corp., Edison, N.J.).

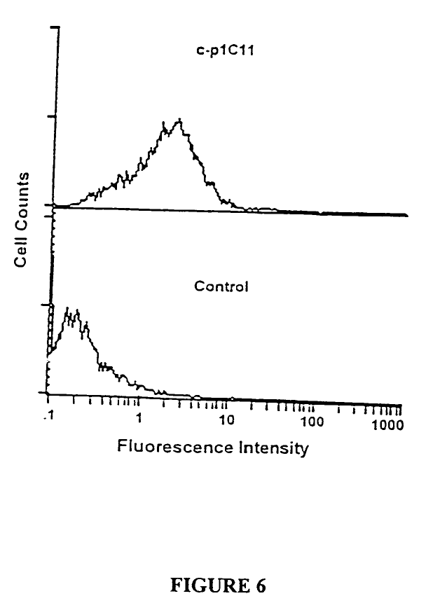

FIG. 6 is a graph showing the FACS analysis of c-p1C11 binding to KDR-expressing HUVEC. As previously seen with the parent scFv p1C11, c-p1C11 binds specifically to KDR expressed on early passage HUVEC.

### Example V(b)

### Quantitative KDR Binding Assay

Various amounts of antibodies were added to KDR-coated 96-well Maxi-sorp microtiter plates (Nunc. Danmark) and incubated at room temperature for 1 h, after which the plates were washed 3 times with PBS containing 0.1% Tween-20. The plates were then incubated at RT for 1 h with 100 ul of mouse anti-E tag antibody-HRP conjugate (Pharmacia Biotech) for the scFv, or rabbit anti-human IgG Fc specific antibody-HRP conjugate (Cappel, Organon Teknika Corp.) for the chimeric IgG. The plates were washed 5 times, TMB peroxidase substrate (KPL, Gaithersburg, Md.) added, and the OD at 450 nm read using a microplate reader (Molecular Device, Sunnyvale, Calif.).

FIG. 5 is a graph showing the direct binding of antibodies to immobilized KDR. C-p1C11 is shown to bind more efficiently to immobilized KDR receptor than the parent scFv.

### Example V(c)

### BIA Core Analysis

The binding kinetics of antibodies to KDR were measured using BIAcore biosensor (Pharmacia Biosensor). KDR-AP fusion protein was immobilized onto a sensor chip, and antibodies or VEGF were injected at concentrations ranging from 25 nM to 200 nM. Sensorgrams were obtained at each concentration and were evaluated using a program, BIA Evaluation 2.0, to determine the rate constants kon and koff. Kd was calculated as the ratio of rate constants koff/kon.

BIAcore analysis reveals that c-p1C11 bind to KDR with higher affinity than the parent scFv (Table 2). The Kd of c-p1C11 is 0.82 nM, compared to 2.1 nM for the scFv. The increased affinity of c-p1C11 is mainly due to a slower dissociation rate (koff) of the bivalent chimeric IgG. It is important to note that the affinity (Kd) of c-p1C11 for binding to KDR is similar to that of the natural ligand VEGF for binding to KDR, which is 0.93 mM as determined in our BIAcore analysis (Table 2).

### Example V(d)

### Competitive VEGF Binding Assay

In the first assay, various amounts of antibodies were mixed with a fixed amount of KDR-AP (50 ng) and incubated at room temperature for 1 h. The mixtures were then transferred to 96-well microtiter plates coated with VEGF₁₆₅ (200 ng/well) and incubated at room temperature for an additional 2 h, after which the plates were washed 5 times and the substrate for AP (p-nitrophenyl phosphate, Sigma) was added to quantify the bound KDR-AP molecules. EC₅₀, i.e., the antibody concentration required for 50% inhibition of KDR binding to VEGF, was then calculated.

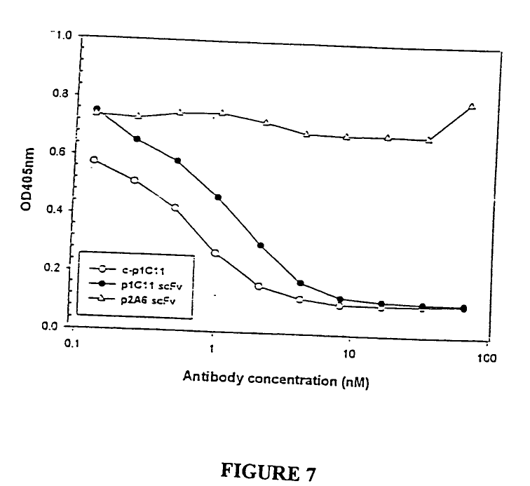

FIG. 7 shows that c-p1C11 block KDR receptor from binding to immobilized VEGF in a dose-dependent manner. The chimeric antibody is more potent in blocking VEGF-KDR interaction with an IC₅₀ (i.e., the antibody concentrations required to inhibit 50% of KDR trom binding to VEGF) of 0.8 nM, compared to that of 2.0 nM for the scFv. The control scFv p2A6 also binds KDR (FIG. 5) but does not block VEGF-KDR interaction (FIG. 7).

In the second assay, various amounts of c-p1C11 antibody or cold VEGF₁₆₅ protein were mixed with a fixed amount of ¹²⁵I labeled VEGF₁₆₅ and added to 96-well microtiter plates coated with KDR receptor. The plates were incubated at room temperature for 2 h, washed 5 times and the amounts of radiolabeled VEGF₁₆₅ that bound to immobilized KDR receptor were counted. Concentrations of c-p1C11 and cold VEGF₁₆₅ required to block 50% of binding of the radiolabeled VEGF to immobilized KDR receptor were determined.

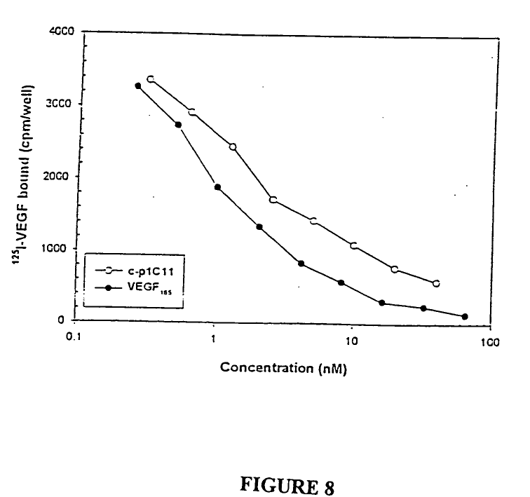

The results of the inhibition of binding of radiolabeled VEGF₁₆₅ is shown in FIG. 8. The data shown are the means of triplicate determinations. c-p1C11 is shown to efficiently compete with ¹²⁵I labeled VEGF for binding to immobilized KDR receptor in a dose-dependent manner. As expected, C225, a chimeric antibody directed against EGF receptor does not bind to KDR receptor or block VEGF-KDR interaction (not shown).

### Example V(e)

### Phosphorylation Assay

Subconfluent HUVEC cells were grown in growth factor depleted EBM-2 medium for 24 to 48 h prior to experimentation. After pretreatment with 50 nM sodium orthovanadate for 30 min, the cells were incubated in the presence or absence of antibodies for 15 min, followed by stimulation with 20 ng/ml of VEGF₁₆₅, or 10 ng/ml of FGF at room temperature for an additional 15 min. The cells were then lysed in lysis buffer (50 nM Tris, 150 mM NaCl, 1% NP-40, 2 mM EDTA, 0.25% sodium deoxycholate, 1 mM PMSF, 1 ug/ml leupeptin, 1 ug/ml pepstatin, 10 ug/ml aprotinin, pH 7.5) and the cell lysate used for both the KDR and MAP kinase phosphorylation assays. The KDR receptor was immunoprecipitated from the cell lysates with Protein A Sepharose beads (Santa Cruz Biotechnology, Inc., CA) coupled to an anti-KDR antibody, Mab 4.13 (ImClone Systems). Proteins were resolved with SDS-PAGE and subjected to Western blot analysis. To detect KDR phosphorylation, blots were probed with an antiphosphotyrosine Mab, PY20 (ICN Biomedicals, Inc. Aurora, Ohio). For the MAP kinase activity assay, cell lysates were resolved with SDS-PAGE followed by Western blot analysis using a phospho-specific MAP kinase antibody (New England BioLabs, Beverly, Mass.). All signals were detected using ECL (Amersham, Arlington Heights, Ill.). In both assays, the blots were reprobed with a polyclonal anti-KDR antibody (ImClone Systems) to assure that equal amount of protein was loaded in each lane of SDS-PAGE gels.

C-p1C11 effectively inhibits VEGF-stimulated phosphorylation of KDR receptor and activation of p44/p42 MAP kineses. In contrast, C225 does not show any inhibition of VEGF-stimulated activation of KDR receptor and MAP kineses. Neither c-p1C11, nor C225 alone has any effects on the activity of KDR receptor and p44/p42 MAP kineses. As previously seen with the scFv p1C11, c-p1C11 does not inhibit FGF-stimulated activation of p44/p42 MAP kineses (not shown). Furthermore, neither scFv p2A6, nor the chimeric IgG form of p2A6 (c-p2A6), inhibits VEGF-stimulated activation of KDR receptor and MAP kineses (not shown).

### Example V(f)

### Anti-Mitogenic Assay

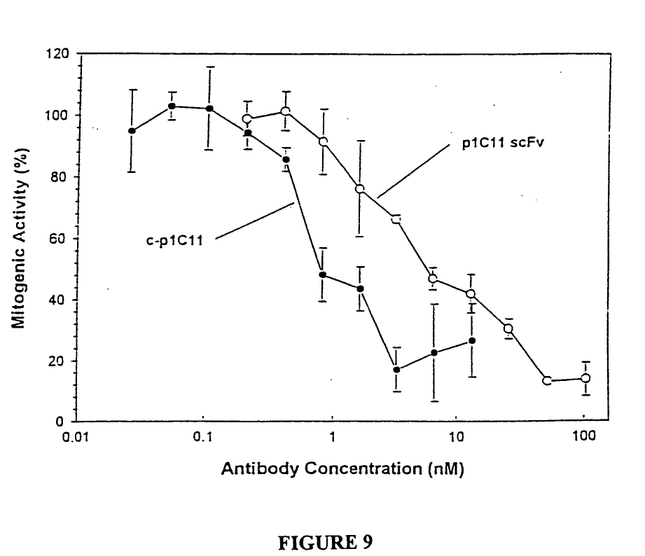

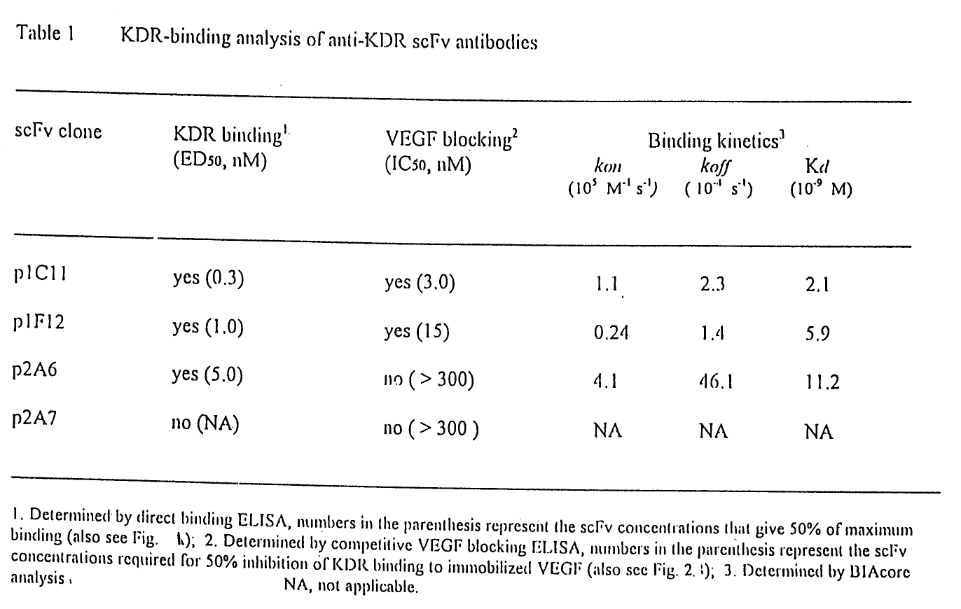

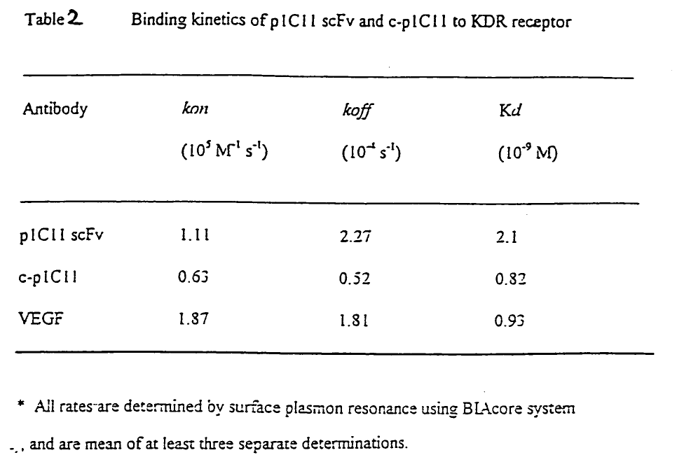

The effect of anti-KDR antibodies on VEGF-stimulated mitogenesis of human endothelial cells was determined with a [³H]-TdR DNA incorporation assay using HUVEC. HUVEC (5×10³ cells/well) were plated into 96-well tissue culture plates in 200 μl of EBM-2 medium without VEGF, bFGF or EGF and incubated at 37 C for 72 h. Various amounts of antibodies were added to duplicate wells and pre-incubated at 37 C for 1 hour, after which VEGF₁₆₅ was added to a final concentration of 16 ng/ml. After 18 hours of incubation, 0.25 μCi of [³H]-TdR was added to each well and incubated for an additional 4 hours. DNA incorporated radioactivity was determined with a scintillation counter. The data shown in FIG. 9 are representative of at least three separate experiments.

Both c-p1C11 and scFv p1C11 effectively inhibit mitogenesis of HUVEC stimulated by VEGF (FIG. 9). C-p1C11 is a stronger inhibitor of VEGF-induced mitogenesis of HUVEC than the parent scFv. The antibody concentrations required to inhibit 50% of VEGF-induced mitogenesis of HUVEC are 0.8 nM for c-p1C11 and 6 nM for the scFv, respectively. As expected, scFv p2A6 does not show any inhibitory effect on VEGF-stimulated endothelial cell proliferation.

1. An immunoglobulin molecule that binds KDR with an affinity comparable to human VEGF, and that neutralizes activation of KDR.

2. An immunoglobulin molecule according to claim 1 wherein the immunoglobulin molecule is selected from the group consisting of a single chain antibody, a diabody, a triabody or an antibody.

3. A single chain antibody that neutralizes activation of KDR, wherein the single chain antibody comprises at least one variable heavy-chain fragment comprising:
CDRH1, having the amino acid sequence shown in SEQ. ID. NO. 1;
CDRH2, having the amino acid sequence shown in SEQ. ID. NO. 2; and
CDRH3, having the amino acid sequence shown in SEQ. ID. NO. 3;
and at least one variable light-chain fragment comprising:
CDRL1, having the amino acid sequence shown in SEQ. ID. NO. 4;
CDRL2, having the amino acid sequence shown in SEQ. ID. NO. 5; and
CDRL3, having the amino acid sequence shown in SEQ. ID. NO. 6.

4. A single chain antibody that neutralizes activation of KDR, wherein the single chain antibody comprises:
at least one variable heavy-chain fragment having the amino acid sequence shown in SEQ. ID. NO. 7; and
at least one variable light-chain fragment having the amino acid sequence shown in SEQ. ID. NO. 8.

5. A nucleic acid molecule that encodes the single chain antibody of claim 3.

6. The nucleic acid molecule of claim 5 which consists of:
the nucleic acid sequence that encodes CDRH1 shown in SEQ. ID. NO. 9;
the nucleic acid sequence that encodes CDRH2 shown in SEQ. ID. NO. 10;
the nucleic acid sequence that encodes CDRH3 shown in SEQ. ID. NO. 11;
the nucleic acid sequence that encodes CDRL1 shown in SEQ. ID. NO. 12;
the nucleic acid sequence that encodes CDRL2 shown in SEQ. ID. NO. 13; and
the nucleic acid sequence that encodes CDRL3 shown in SEQ. ID. NO. 14.

7. A nucleic acid molecule that encodes the single chain antibody of claim 4.

8. The nucleic acid molecule of claim 7 which consists of:
the nucleic acid sequence that encodes variable heavy-chain fragment shown in SEQ. ID. NO. 15; and
the nucleic acid sequence that encodes variable light-chain fragment shown in SEQ. ID. NO. 16.

9. The single chain antibody of claim 3 wherein the variable heavy-chain fragment and the variable light-chain fragment are covalently linked by at least one peptide linker.

10. The single chain antibody of claim 9 wherein the peptide linker comprises at least 15 amino acids.

11. The single chain antibody of claim 9 wherein the peptide linker comprises the amino acid sequence shown in SEQ. ID. NO. 17.

12. A nucleic acid molecule that encodes the peptide linker of claim 11.

13. The nucleic acid molecule of claim 12 which consists of the nucleic acid sequence that encodes the peptide linker shown in SEQ. ID. NO. 18.

14. A diabody that neutralizes activation of KDR, wherein the diabody comprises at least one variable heavy-chain fragment comprising:
CDRH1, having the amino acid sequence shown in SEQ. ID. NO. 1;
CDRH2, having the amino acid sequence shown in SEQ. ID. NO. 2; and
CDRH3, having the amino acid sequence shown in SEQ. ID. NO. 3;
and at least one variable light-chain fragment comprising:
CDRL1, having the amino acid sequence shown in SEQ. ID. NO. 4;
CDRL2, having the amino acid sequence shown in SEQ. ID. NO. 5; and
CDRL3, having the amino acid sequence shown in SEQ. ID. NO. 6.

15. A diabody that neutralizes activation of KDR, wherein the diabody comprises
a variable heavy-chain fragment having the amino acid sequence shown in SEQ. ID. NO. 7; and
a variable light-chain fragment having the amino acid sequence shown in SEQ. ID. NO. 8.

16. A nucleic acid molecule that encodes the diabody of claim 14.

17. The nucleic acid molecule of claim 16 which consists of:
the nucleic acid sequence that encodes CDRH1 shown in SEQ. ID. NO. 9;
the nucleic acid sequence that encodes CDRH2 shown in SEQ. ID. NO. 10;
the nucleic acid sequence that encodes CDRH3 shown in SEQ. ID. NO. 11;
the nucleic acid sequence that encodes CDRL1 shown in SEQ. ID. NO. 12;
the nucleic acid sequence that encodes CDRL2 shown in SEQ. ID. NO. 13; and
the nucleic acid sequence that encodes CDRL3 shown in SEQ. ID. NO. 14.

18. A nucleic acid molecule that encodes the diabody of claim 15.

19. The nucleic acid molecule of claim 18 which consists of:
the nucleic acid sequence that encodes variable heavy-chain fragment shown in SEQ. ID. NO. 15; and
the nucleic acid sequence that encodes variable light-chain fragment shown in SEQ. ID. NO. 16.

20. The diabody of claim 14 wherein the variable heavy-chain fragment and the variable light-chain fragment are covalently linked by at least one peptide linker.

21. The diabody of claim 20 wherein the peptide linker comprises at least 5 amino acids and no more than 10 amino acids.

22. The diabody claim 21 wherein the peptide linker comprises the amino acid sequence shown in SEQ. ID. NO. 19.

23. A nucleic acid molecule that encodes the peptide linker of claim 22.

24. The nucleic acid molecule of claim 23 which consists of the nucleic acid sequence that encodes for the peptide linker shown in SEQ. ID. NO. 20.

25. The diabody of claim 14, wherein said diabody is monospecific.

26. The diabody of claim 14, wherein said diabody is bispecific and wherein the diabody binds to at least one epitope on KDR.

27. A triabody that neutralizes activation of KDR, wherein the triabody comprises at least one variable heavy-chain fragment comprising:
CDRH1, having the amino acid sequence shown in SEQ. ID. NO. 1;
CDRH2, having the amino acid sequence shown in SEQ. ID. NO. 2; and
CDRH3, having the amino acid sequence shown in SEQ. ID. NO. 3;
and at least one variable light-chain fragment comprising:
CDRL1, having the amino acid sequence shown in SEQ. ID. NO. 4;
CDRL2, having the amino acid sequence shown in SEQ. ID. NO. 5; and
CDRL3, having the amino acid sequence shown in SEQ. ID. NO. 6.

28. A triabody that neutralizes activation of KDR, wherein the triabody comprises
at least one variable heavy-chain fragment having the amino acid sequence shown in SEQ. ID. NO. 7; and
at least one variable light-chain fragment having the amino acid sequence shown in SEQ. ID. NO. 8.

29. A nucleic acid molecule that encodes the triabody of claim 27.

30. The nucleic acid molecule of claim 29 which consists of:
the nucleic acid sequence that encodes CDRH1 shown in SEQ. ID. NO. 9;
the nucleic acid sequence that encodes CDRH2 shown in SEQ. ID. NO. 10;
the nucleic acid sequence that encodes CDRH3 shown in SEQ. ID. NO. 11;
the nucleic acid sequence that encodes CDRL1 shown in SEQ. ID. NO. 12;
the nucleic acid sequence that encodes CDRL2 shown in SEQ. ID. NO. 13; and
the nucleic acid sequence that encodes CDRL3 shown in SEQ. ID. NO. 14.

31. A nucleic acid molecule that encodes the triabody of claim 28.

32. The nucleic acid molecule of claim 31 which consists of:
the nucleic acid sequence that encodes variable heavy-chain fragment shown in SEQ. ID. NO. 15; and
the nucleic acid sequence that encodes variable light-chain fragment shown in SEQ. ID. NO. 16.

33. The triabody of claim 27, wherein said triabody is monospecific.

34. The triabody of claim 27, wherein said triabody is bispecific and wherein the triabody binds to at least one epitope on KDR.

35. The triabody of claim 27, wherein said triabody is trispecific and wherein the triabody binds to at least one epitope on KDR.

36. An antibody that neutralizes activation of KDR, wherein the antibody comprises at least one variable heavy-chain fragment comprising:
CDRH1, having the amino acid sequence shown in SEQ. ID. NO. 1;
CDRH2, having the amino acid sequence shown in SEQ. ID. NO. 2; and
CDRH3, having the amino acid sequence shown in SEQ. ID. NO. 3;
and at least one variable light-chain fragments comprising:
CDRL1, having the amino acid sequence shown in SEQ. ID. NO. 4;
CDRL2, having the amino acid sequence shown in SEQ. ID. NO. 5; and
CDRL3, having the amino acid sequence shown in SEQ. ID. NO. 6.

37. An antibody that neutralizes activation of KDR wherein the antibody comprises:
a variable heavy-chain fragment having the amino acid sequence shown in SEQ. ID. NO. 7; and
a variable light-chain fragment having the amino acid sequence shown in SEQ. ID. NO. 8.

38. A nucleic acid molecule that encodes an antibody that neutralizes activation of KDR which comprises:
the nucleic acid sequence that encodes CDRH1 shown in SEQ. ID. NO. 9;
the nucleic acid sequence that encodes CDRH2 shown in SEQ. ID. NO. 10;
the nucleic acid sequence that encodes CDRH3 shown in SEQ. ID. NO. 11;
the nucleic acid sequence that encodes CDRL1 shown in SEQ. ID. NO. 12;
the nucleic acid sequence that encodes CDRL2 shown in SEQ. ID. NO. 13; and
the nucleic acid sequence that encodes CDRL3 shown in SEQ. ID. NO. 14.

39. A nucleic acid molecule that encodes an antibody that neutralizes activation of KDR which comprises:
the nucleic acid sequence that encodes the variable heavy-chain fragment shown in SEQ. ID. NO. 15; and
the nucleic acid sequence that encodes the variable light-chain fragment shown in SEQ. ID. NO. 16.

40. A chimerized antibody comprising the antibody of claim 36.

41. A humanized antibody comprising the antibody of claim 36.

42. A method of making immunoglobulin molecules that bind KDR with an affinity comparable to human VEGF, and that neutralize activation of KDR comprising:
inserting a nucleic acid sequence that encodes the immunoglobulin molecule into a host cell,
and expressing that nucleic acid sequence.

43. A method of neutralizing the activation of KDR comprising:
administering to a mammal an effective amount of an immunoglobulin molecule that binds KDR with an affinity comparable to human VEGF, and that neutralizes activation of KDR.

44. A method of reducing tumor growth comprising:
administering to a mammal an effective amount of an immunoglobulin molecule that binds KDR with an affinity comparable to human VEGF, and that neutralizes activation of KDR.

45. A method of inhibiting angiogenesis comprising:
administering to a mammal an effective amount of an immunoglobulin molecule that binds KDR with an affinity comparable to human VEGF, and that neutralizes activation of KDR.

46. A method of making single chain antibodies that bind KDR with an affinity comparable to human VEGF and that neutralize activation of KDR comprising:
inserting a nucleic acid sequence that encodes the single chain antibody into a host cell,
and expressing that nucleic acid sequence.

47. A method of neutralizing the activation of KDR comprising:
administering to a mammal an effective amount of a single chain antibody that binds KDR with an affinity comparable to human VEGF, and that neutralizes activation of KDR.

48. A method of reducing tumor growth comprising:
administering to a mammal an effective amount of a single chain antibody that binds KDR with an affinity comparable to human VEGF, and that neutralizes activation of KDR.

49. A method of inhibiting angiogenesis comprising:
administering to a mammal an effective amount of a single chain antibody that binds KDR with an affinity comparable to human VEGF, and that neutralizes activation of KDR.

50. A method of making diabodies that bind KDR with an affinity comparable to human VEGF and that neutralize activation of KDR comprising:
inserting a nucleic acid sequence that encodes the diabody into a host cell,
and expressing that nucleic acid sequence.

51. A method of neutralizing the activation of KDR comprising:
administering to a mammal an effective amount of a diabody that binds KDR with an affinity comparable to human VEGF, and that neutralizes activation of KDR.

52. A method of reducing tumor growth comprising:
administering to a mammal an effective amount of a diabody that binds KDR with an affinity comparable to human VEGF, and that neutralizes activation of KDR.

53. A method of inhibiting angiogenesis comprising:
administering to a mammal an effective amount of a diabody that binds KDR with an affinity comparable to human VEGF, and that neutralizes activation of KDR.

54. A method of making triabodies that bind KDR with an affinity comparable to human VEGF and that neutralize activation of KDR comprising:
inserting a nucleic acid sequence that encodes the triabody into a host cell,
and expressing that nucleic acid sequence.

55. A method of neutralizing the activation of KDR comprising:
administering to a mammal an effective amount of a triabody that binds KDR with an affinity comparable to human VEGF, and that neutralizes activation of KDR.

56. A method of reducing tumor growth comprising:
administering to a mammal an effective amount of a triabody that binds KDR with an affinity comparable to human VEGF, and that neutralizes activation of KDR.

57. A method of inhibiting angiogenesis comprising:
administering to a mammal an effective amount of a triabody that binds KDR with an affinity comparable to human VEGF, and that neutralizes activation of KDR.

58. A method of making antibodies that bind KDR with an affinity comparable to human VEGF and that neutralize activation of KDR comprising:
inserting a nucleic acid sequence that encodes the antibody into a host cell,
and expressing that nucleic acid sequence.

59. A method of neutralizing the activation of KDR comprising:
administering to a mammal an effective amount of an antibody that binds KDR with an affinity comparable to human VEGF, and that neutralizes activation of KDR.

60. A method of reducing tumor growth comprising:
administering to a mammal an effective amount of an antibody that binds KDR with an affinity comparable to human VEGF, and that neutralizes activation of KDR.

61. A method of inhibiting angiogenesis comprising:
administering to a mammal an effective amount of an antibody that binds KDR with an affinity comparable to human VEGF, and that neutralizes activation of KDR.


SEQUENCE LISTING (40 sequences)

SEQ ID NO: 1 - protein, 10 aa, Mus musculus
GFNIKDFYMH

SEQ ID NO: 2 - protein, 17 aa, Mus musculus
WIDPENGDSGYAPKFQG

SEQ ID NO: 3 - protein, 8 aa, Mus musculus
YYGDYEGY

SEQ ID NO: 4 - protein, 10 aa, Mus musculus
SASSSVSYMH

SEQ ID NO: 5 - protein, 7 aa, Mus musculus
STSNLAS

SEQ ID NO: 6 - protein, 9 aa, Mus musculus
QQRSSYPFT

SEQ ID NO: 7 - protein, 117 aa, Mus musculus
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSS

SEQ ID NO: 8 - protein, 108 aa, Mus musculus
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 9 - DNA, 30 bp, Mus musculus
ggcttcaacattaaagacttctatatgcac

SEQ ID NO: 10 - DNA, 51 bp, Mus musculus
tggattgatcctgagaatggtgattctggttatgccccgaagttccagggc

SEQ ID NO: 11 - DNA, 24 bp, Mus musculus
tactatggtgactacgaaggctac

SEQ ID NO: 12 - DNA, 30 bp, Mus musculus
agtgccagctcaagtgtaagttacatgcac

SEQ ID NO: 13 - DNA, 21 bp, Mus musculus
agcacatccaacctggcttct

SEQ ID NO: 14 - DNA, 27 bp, Mus musculus
cagcaaaggagtagttacccattcacg

SEQ ID NO: 15 - DNA, 351 bp, Mus musculus
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctggttatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 16 - DNA, 324 bp, Mus musculus
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaacgggcg

SEQ ID NO: 17 - protein, 15 aa, Artificial Sequence
  peptide linker
GGGGSGGGGSGGGGS

SEQ ID NO: 18 - DNA, 45 bp, Artificial Sequence
  nucleic acid encoding peptide linker
ggtggaggcggttcaggcggaggtggctctggcggtggcggatcg

SEQ ID NO: 19 - protein, 10 aa, Artificial Sequence
  peptide linker
GGGGSGGGGS

SEQ ID NO: 20 - DNA, 15 bp, Artificial Sequence
  nucleic acid encoding peptide linker
ggtggaggcggttca

SEQ ID NO: 21 - protein, 5 aa, Artificial Sequence
  peptide linker
GGGGS

SEQ ID NO: 22 - protein, 17 aa, Mouse
WIDPENGDSDYAPKFQG

SEQ ID NO: 23 - protein, 117 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSS

SEQ ID NO: 24 - protein, 106 aa, Mouse
DIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 25 - DNA, 51 bp, Mouse
tggattgatcctgagaatggtgattctgattatgccccgaagttccagggc

SEQ ID NO: 26 - DNA, 351 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctgattatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctca

SEQ ID NO: 27 - DNA, 318 bp, Mouse
gacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaa

SEQ ID NO: 28 - protein, 240 aa, Mouse
QVKLQQSGAELVGSGASVKLSCTTSGFNIKDFYMHWVKQRPEQGLEWIGWIDPENGDSGYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 29 - protein, 238 aa, Mouse
QVKLQQSGAELVGSGASVKLSCTTSGFNIKDFYMHWVKQRPEQGLEWIGWIDPENGDSDYAPKFQGKATMTADSSSNTAYLQLSSLTSEDTAVYYCNAYYGDYEGYWGQGTTVTVSSGGGGSGGGGSGGGGSDIELTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIK

SEQ ID NO: 30 - DNA, 720 bp, Mouse
caggtcaagctgcagcagtctggggcagagcttgtggggtcaggggcctcagtcaaattgtcctgcacaacttctggcttcaacattaaagacttctatatgcactgggtgaagcagaggcctgaacagggcctggagtggattggatggattgatcctgagaatggtgattctggttatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctcaggtggaggcggttcaggcggaggtggctctggcggtggcggatcggacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaacgggcg

SEQ ID NO: 31 - DNA, 714 bp, Mouse
caggtcaagctgcagcagtctggggcagagcttgtggggtcaggggcctcagtcaaattgtcctgcacaacttctagcttcaacattaaagacttctatatgcactgggtgaagcagaggcctgaacagggcctggagtggattggatggattgatcctgagaatggtgattctgattatgccccgaagttccagggcaaggccaccatgactgcagactcatcctccaacacagcctacctgcagctcagcagcctgacatctgaggacactgccgtctattactgtaatgcatactatggtgactacgaaggctactggggccaagggaccacggtcaccgtctcctcaggtggaggcggttcaggcggaggtagctctggcggtggcggatcggacatcgagctcactcagtctccagcaatcatgtctgcatctccaggggagaaggtcaccataacctgcagtgccagctcaagtgtaagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccattcacgttcggctcggggaccaagctggaaataaaa

SEQ ID NO: 32 - protein, 19 aa, Mouse
  leader peptide
MGWSCLILFLVATATGVHS

SEQ ID NO: 33 - DNA, 57 bp, Mouse
atgggatggtcatgtctcatcctttttctagtagcaactgcaactggagtacattca

SEQ ID NO: 34 - protein, 19 aa, Mouse
  leader peptide
MGWSCIILFLVATATGVHS

SEQ ID NO: 35 - DNA, 57 bp, Mouse
atgggatggtcatgtatcatcctttttctagtagcaactgcaactggagtacattca

SEQ ID NO: 36 - DNA, 42 bp, Artificial Sequence
  amplification primer
ctagtagcaactgcaactggagtacattcagacatcgagctc

SEQ ID NO: 37 - DNA, 36 bp, Artificial Sequence
  amplification primer
tcgatctagaaggatccactcacgttttatttccag

SEQ ID NO: 38 - DNA, 42 bp, Artificial Sequence
  amplification primer
ctagtagcaactgcaactggagtacattcacaggtcaagctg

SEQ ID NO: 39 - DNA, 30 bp, Artificial Sequence
  amplification primer
tcgaaggatccactcacctgaggagacggt

SEQ ID NO: 40 - DNA, 51 bp, Artificial Sequence
  amplification primer
ggtcaaaagcttatgggatggtcatgtatcatcctttttctagtagcaact